(19) United States
(12) Patent Application Publication
(10) Pub. No.: US 2025/0235520 A1
(43) Pub. Date: Jul. 24, 2025

(54) METHODS FOR REVERSING A SUPPRESSED IMMUNE STATE TO INCREASE SURVIVAL IN A SUBJECT

(71) Applicant: Greenwich LifeSciences, Inc., Stafford, TX (US)

(72) Inventors: Snehal S. Patel, Sugar Land, TX (US); Jaye Thompson, Woodlands, TX (US)

(21) Appl. No.: 18/839,849

(22) PCT Filed: Mar. 8, 2023
(86) PCT No.: PCT/US2023/014811

§ 371 Date: Aug. 20, 2024

Related U.S. Application Data
(60) Provisional application No. 63/317,601, filed on Mar. 8, 2022.
(60) Provisional application No. 63/329,409, filed on Apr. 9, 2022.

(51) Int. Cl. A61K 39/00; A61P 35/00; A61P 37/04

(52) U.S. Cl. CPC A61K 39/001106; A61P 35/00; A61P 37/04; A61K 2039/55522; A61K 2039/812

(57) ABSTRACT

The present disclosure provides methods for using a peptide of an oncogenic protein to reverse immune suppression, cause stronger immune responses, lower cancer recurrence rates, and lengthen disease-free survival. The suppressed immune state can be due to prior treatments, including but not limited to surgery, chemotherapy, antibodies targeting the oncogenic protein, high levels of expression of the oncogenic protein, or combination thereof. It has been demonstrated that if GM-CSF is administered to patients without the peptide from the oncogenic protein, such as in the control group of a clinical study, the immune response to GM-CSF alone is lower when compared to patients who are not treated with an antibody to the oncogenic protein and who do not have high levels of expression of the oncogenic protein: their immune state is suppressed, recurrence rate is higher, and disease free survival is lower, which are all reversed by the addition of the peptide.

### CROSS-REFERENCE TO RELATED APPLICATIONS

This application claims the benefit of U.S. Provisional Application Ser. No. 63/317,601, filed Mar. 8, 2022, and U.S. Provisional Application Ser. No. 63/329,409, filed Apr. 9, 2022, each of which are incorporated by reference herein in their entirety.

### SEQUENCE LISTING

This application contains a Sequence Listing electronically submitted via EFS-Web to the United States Patent and Trademark Office as an ASCII text file entitled “0629.000003WO01.xml” having a size of 2 kilobytes and created on Mar. 8, 2023. The information contained in the Sequence Listing is incorporated by reference herein.

### BACKGROUND

HER2/neu (human epidermal growth factor receptor 2, also referred to herein as HER2) protein is a cell surface receptor protein that is expressed in a variety of common cancers, including 75% of breast cancers. In breast cancers the protein can be expressed at low (1+), intermediate (2+), and high (3+ or over-expressor or positive) levels. Resection of the tumor by surgery is helpful in treatment, and further therapy is typically recommended. There are a number of therapies approved for HER2/neu-expressing breast cancers. For instance, a HER2/neu-expressing subject can receive trastuzumab (Herceptin®) or a trastuzumab-based derivative before surgery or in the first year after surgery as a follow up therapy to reduce the likelihood of recurrence. However, some patients do not respond to the trastuzumab-based therapy and recur within five years of the surgery or thereafter.

### SUMMARY OF THE APPLICATION

GP2 is a 9 amino acid transmembrane peptide of the HER2/neu protein and is a natural cancer antigen. GP2, in combination with granulocyte-macrophage colony-stimulating factor (GM-CSF), which combined are called GLSI-100, is a candidate therapy to reduce cancer recurrence in breast cancer patients with tumors expressing any degree of HER2/neu and to therefore increase disease free survival. It was found that GLSI-100 safely elicited a potent immune response, as evidenced by injection site reactions (ISRs), which led to a reduced recurrence rate of 0% over 5 years of follow-up in HER2 3+ or positive patients and which led to a correspondingly increased disease free survival rate of 100%. These patients received a standard course of trastuzumab after surgery and were then treated with GLSI-100. The injection site reactions of HER2 3+ patients treated with GLSI-100 were significantly larger than those of the GM-CSF alone control group providing further evidence of specific GP2 immune response and a boosting of the immune response produced specifically and solely by adding GP2. The lower immune responses of the GM-CSF alone HER2 positive patients is suggestive of a reduced immune state compared to HER2 low and intermediate expressors (HER2 1+ and HER2 2+) treated with either GLSI-100 or GM-CSF alone, who had ISRs similar to HER2 positive patients treated with GLSI-100. HER2 low and intermediate expressors were not treated with trastuzumab. Thus, the addition of GP2 alleviates the reduced immune state in HER2 positive patients, which may be caused by HER2 positivity and/or prior treatment with trastuzumab. Trastuzumab treatment could also include derivatives of trastuzumab such as antibody drug conjugates or other antibodies targeting the extracellular portion of the HER2/neu protein. The ISRs resulting from GLSI-100 administration correlate to and can be used to complement or replace a delayed-type hypersensitivity (DTH) immune response to GP2. Thus, as described herein an immune response such as, but not limited to, a positive DTH immune response to a peptide or an ISR, or a combination thereof, can be indicative of a subject's immune state, including whether the subject is experiencing an immune response and is thus less likely to have a recurrence of a cancer and is more likely to experience longer disease-free survival. The peptide can be one that is a peptide of a protein correlated with a cancer, such as a peptide of an oncogenic protein. In one embodiment, the peptide is a portion of Her2/neu, such as GP2.

Terms used herein will be understood to take on their ordinary meaning in the relevant art unless specified otherwise. Several terms used herein, and their meanings are set forth below.

“Relapse” and “recurrence” are used interchangeably herein, and refer to the diagnosis of return, or signs and symptoms of return of breast cancer after a period of improvement or remission. Cancer recurrence can be diagnosed using standard of care methods such as patient follow-up visits, blood test, mammogram, and imaging.

As used herein, “oncoprotein” and “oncogenic protein” are used interchangeably and refer to a protein that is correlated to cancer in a human. Oncoproteins are encoded by oncogenes. Oncogenes are the mutated forms of normal cellular genes (proto-oncogenes). The protein products of proto-oncogenes stimulate cell division and/or inhibit cell death. Proto-oncogenes can be likened to the gas pedal in a car. Normally, internal and external signals strictly regulate the activity of the proto-oncogenes, but oncogenes are defective and are ‘on’ even when they do not receive appropriate signals. Oncogenes also help cells to ignore negative signals that would prevent a healthy cell from dividing. Oncogenes can cause cells to divide continuously even in the absence of any pro-growth signals. The following list describes different cellular roles for a few of the many known oncogenes. HER-2/neu: HER-2/neu encodes for a cell surface receptor that can stimulate cell division, the HER-2/neu gene is amplified in up to 30% of human breast cancers; RAS: the Ras gene products are involved in kinase signaling pathways that ultimately control transcription of genes, regulating cell growth and differentiation, overexpression and amplification of RAS can lead to continuous cell proliferation; MYC: the Myc protein is a transcription factor and controls expression of several genes, Myc is thought to be involved in avoiding the cell death mechanism, MYC oncogenes may be activated by gene rearrangement or amplification; SRC: SRC was the first oncogene discovered, the Src protein is a tyrosine kinase which regulates cell activity; hTERT: hTERT codes for an enzyme (telomerase) that maintains chromosome ends, in most normal cells telomerase is only present during fetal development, activation of hTERT in adult cells gives them the ability to divide indefinitely; BCL-2: the Bcl-2 protein works to prevent cell death (apoptosis), overexpression of BCL-2 allows continued division of mutated cells. An oncogenic protein can be one that is expressed by non-cancer cells but at higher levels in cancer cells, or one that is not expressed in non-cancer cells but expressed in cancer cells. Examples of oncogenic proteins are shown in Table 1 (also on the world wide web at cancerquest.org/cancer-biology/cancer-genes).

TABLE 1

| Oncogene | Function/Activation | Cancer* |
|---|---|---|
| ABL1 | Promotes cell growth through | Chronic myelogenous |
| | tyrosine kinase activity | leukemia |
| AFF4/MLLT11 | Fusion affects the MLLT11 | Acute leukemias |
| | transcription | |
| | factor/methyltransferase. MLLT11 | |
| | is also called HRX, ALL1 and | |
| | HTRX1 | |
| AKT2 | Encodes a protein-serine/threonine | Ovarian cancer |
| | kinase | |
| ALK | Encodes a receptor tyrosine kinase | Lymphomas |
| ALK/NPM | Translocation creates fusion | Large cell lymphomas |
| | protein with nucleophosmin(npm) | |
| RUNX1 (AML1) | Encodes a transcription factor | Acute myeloid leukemia |
| RUNX1/MTG8(ETO) | New fusion protein created by | Acute leukemias |
| | translocation | |
| AXL | Encodes a receptor tyrosine kinase | Hematopoietic cancers |
| BCL-2, 3, 6 | Block apoptosis (programmed cell | B-cell lymphomas and |
| | death) | leukemias |
| BCR/ABL | New protein created by fusion of | Chronic myelogenous and |
| | bcr and abl triggers unregulated | acute lymphotic leukemia |
| | cell growth | |
| MYC (c-MYC) | Transcription factor that promotes | Leukemia; breast, stomach, |
| | cell proliferation and DNA | lung, cervical, and colon |
| | synthesis | carcinomas; neuroblastomas |
| | | and glioblastomas |
| MCF2 (DBL) | Guanine nucleotide exchange | Diffuse B-cell lymphoma |
| | factor | |
| DEK/NUP214 | New protein created by fusion | Acute myeloid leukemia |
| TCF3/PBX1 | New protein created by fusion | Acute pre B-cell leukemia; |
| | | TCF3 also called E2A |
| EGFR | Cell surface receptor that triggers | Squamous cell carcinoma, |
| | cell growth through tyrosine kinase | glioblastomas, lung cancer |
| | activity | |
| MLLT11 | Fusion protein created by a | Acute leukemias |
| | translocation t(11; 19). | |
| ERG/FUS | Fusion protein created by t(16; 21) | Myeloid leukemia |
| | translocation. The erg protein is a | |
| | transcription factor. | |
| ERBB2 | Cell surface receptor that triggers | Breast, salivary gland, and |
| | cell growth through tyrosine kinase | ovarian carcinomas |
| | activity; also known as HER2 or | |
| | neu | |
| ETS1 | Transcription factor | Lymphoma |
| EWSR1/FLI1 | Fusion protein created by t(11; 22) | Ewing Sarcoma |
| | translocation. | |
| CSF1R | Tyrosine kinase | Sarcoma |
| FOS | Transcription factor for API | Osteosarcoma |
| FES | Tyrosine kinase | Sarcoma |
| GLI1 | Transcription factor | Glioblastoma |
| GNAS (GSP) | Membrane associated G protein | Thyroid carcinoma |
| HER2/neu | Overexpression of signaling kinase | Breast, ovarian, gastric, and |
| | due to gene amplification | cervical carcinomas |
| TLX1 | Transcription factor; aka HOX11 | Acute T-cell leukemia |
| FGF4 | Encodes fibroblast growth factor; | Breast and squamous cell |
| | aka HST1 | carcinomas |
| IL3 | Cell signaling molecule | Acute pre B-cell leukemia |
| FGF3 (INT-2) | Encodes a fibroblast growth factor | Breast and squamous cell |
| | | carcinomas |
| JUN | Transcription factor for API | Sarcoma |
| KIT | Tyrosine kinase | Sarcoma |
| FGF4 (KS3) | Herpes virus encoded growth | Kaposi's sarcoma |
| | factor | |
| K-SAM | Fibroblast growth factor receptor | Stomach carcinomas |
| AKAP13 | Guanine nucleotide exchange | Myeloid leukemias |
| | factor; aka LBC | |
| LCK | Tyrosine kinase | T-cell lymphoma |
| LMO1, LMO2 | Transcription factors | T-cell lymphoma |
| MYCL | Transcription factor | Lung carcinomas |
| LYL1 | Transcription factor | Acute T-cell leukemia |
| NFKB2 | Transcription factor. Also | B-cell lymphoma |
| | called LYT-10 | |
| NFKB2/Cα1 | Fusion protein formed by | |
| | the (10; 14)(q24; q32) translocation | |
| | of NFKB2 next to the C alpha 1 | |
| | immunoglobulin locus. | |
| MAS1 | Angiotensin receptor | Mammary carcinoma |
| MDM2 | Encodes a protein that inhibits and | Sarcomas |
| | leads to the degradation of p53 | |
| MLLT11 | Transcription | Acute myeloid leukemia |
| | factor/methyltransferase (also | |
| | called HRX and ALL1) | |
| MOS | Serine/threonine kinase | Lung cancer |
| RUNX1T1 | Fusion of transcription repressor to | Acute leukemias |
| | factor to a transcription factor. | |
| | Also known as MTG8 and AML1- | |
| | MTG8 | |
| MYB | Transcription factor | Colon carcinoma and |
| | | leukemias |
| MYH11/CBFB | New protein created by fusion of | Acute myeloid leukemia |
| | transcription factors via an | |
| | inversion in chromosome 16. | |
| NEU | Tyrosine kinase. Also called | Glioblastomas, and squamous |
| | ERBB2 or HER2 | cell carcinomas |
| MYCN | Cell proliferation and DNA | Neuroblastomas, |
| | synthesis | retinoblastomas, and lung |
| | | carcinomas |
| MCF2L (OST) | Guanine nucleotide exchange | Osteosarcomas |
| | factor | |
| PAX-5 | Transcription factor | Lympho-plasmacytoid B-cell |
| | | lymphoma |
| PBX1/E2A | Fusion protein formed via t(1; 19) | Acute pre B-cell leukemia |
| | translocation. Transcription factor | |
| PIM1 | Serine/threonine kinase | T-cell lymphoma |
| CCND1 | Encodes cyclin D1. Involved in | Breast and squamous cell |
| | cell cycle regulation. Also called | carcinomas |
| | PRAD1 | |
| RAF1 | Serine/threonine kinase | Many cancer types |
| RARA/PML | Fusion protein caused by t(15; 17) | Acute premyelocytic leukemia |
| | translocation. Retinoic acid | |
| | receptor. | |
| HRAS | G-protein. Signal transduction. | Bladder carcinoma |
| KRAS | G-protein. Signal transduction | Lung, ovarian, and bladder |
| | | carcinoma |
| NRAS | G-protein. Signal transduction | Breast carcinoma |
| REL/NRG | Fusion protein formed by deletion | B-cell lymphoma |
| | in chromosome 2. Transcription | |
| | factor. | |
| RET | Cell surface receptor. Tyrosine | Thyroid carcinomas, multiple |
| | kinase | endocrine neoplasia type 2 |
| RHOM1, RHOM2 | Transcription factors aka LMO1 | Acute T-cell leukemia |
| | and LMO2 | |
| ROS1 | Tyrosine kinase | Sarcoma |
| SKI | Transcription factor | Carcinomas |
| SIS (aka PDGFB) | Growth factor | Glioma, fibrosarcoma |
| SET/CAN | Fusion protein formed by | Acute myeloid leukemia |
| | rearrangement of chromosome 9. | |
| | Protein localization | |
| SRC | Tyrosine kinase | Sarcomas |
| TAL1, TAL2 | Transcription factor. TAL1 is also | Acute T-cell leukemia |
| | called SCL | |
| NOTCH1 (TAN1) | Altered form of Notch (a cellular | Acute T-cell leukemia |
| | receptor) formed by t(7; 9) | |
| | translocation | |
| TIAM1 | Guanine nucleotide exchange | T-lymphoma |
| | factor | |
| TSC2 | GTPase activator | Renal and brain tumors |
| NTRK1 | Receptor tyrosine kinase | Colon and thyroid carcinomas |

*The cancer types listed in this column are those that are predominantly associated with each oncogene but this is not a complete list.

As used herein, a “peptide” refers to a portion of protein, such as a protein that correlates with a cancer, e.g., an oncoprotein, that is of a size sufficient to generate an immune response. A peptide can be at least 6 amino acids, at least 9 amino acids, or at least 12 amino acids in length.

Unless otherwise specified, “a,” “an,” “the,” and “at least one” are used interchangeably and mean one or more than one.

As used in this specification and the appended claims, the term “or” is generally employed in its sense including “and/or” unless the content clearly dictates otherwise. The term “and/or” means one or all of the listed elements or a combination of any two or more of the listed elements. The use of “and/or” in some instances does not imply that the use of “or” in other instances may not mean “and/or.”

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

As used herein, “have,” “has,” “having,” “include,” “includes,” “including,” “comprise,” “comprises,” “comprising” or the like are used in their open ended inclusive sense, and generally mean “include, but not limited to,” “includes, but not limited to,” or “including, but not limited to.”

It is understood that wherever embodiments are described herein with the language “have,” “has,” “having,” “include,” “includes,” “including,” “comprise,” “comprises,” “comprising” and the like, otherwise analogous embodiments described in terms of “consisting of” and/or “consisting essentially of” are also provided. The term “consisting of” means including, and limited to, whatever follows the phrase “consisting of.” That is, “consisting of” indicates that the listed elements are required or mandatory, and that no other elements may be present. The term “consisting essentially of” indicates that any elements listed after the phrase are included, and that other elements than those listed may be included provided that those elements do not interfere with or contribute to the activity or action specified in the disclosure for the listed elements.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

Throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

In the description herein particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The following detailed description of illustrative embodiments of the present disclosure may be best understood when read in conjunction with the following drawings.

FIG. 1 shows HER2 3+subjects that completed the primary immunization series after treatment with trastuzumab. 100.0% refers to the probability of disease-free survival for subjects treated with GLSI-100, 89.4% refers to the probability of disease-free survival for placebo subjects treated with GM-CSF. The insert describes the median orthogonal mean of injection site reactions (ISRs) over time for the HER2 3+subjects treated with GLSI-100 (upper trace) or the HER2 3+ placebo subjects treated with GM-CSF alone (lower trace). Median Orthogonal Mean (Y-axis) of the insert is the median orthogonal mean of Injection Site Reactions (ISRs). HER2+ and HER2 positive are equivalent to HER2 3+; HER2- and HER2 negative are equivalent to HER2 1+ and HER2 2+. The two rows of numbers below the X-axis entitled “GP2” and “Placebo” refer to the number of patients in each group at each tested timepoint. Trastuzumab treated HER2 3+ patients receiving GLSI-100 having the non-suppressed immune state (upper trace of the insert) were the patients with 100% disease-free survival during the 5-year follow up (upper trace).

FIG. 2 shows correlation between month 6 DTH and ISR at dose 6 injection in GLSI-100 treated subjects. A single outlier was excluded from this analysis due to the influence of this point.

FIG. 3 shows correlation between baseline DTH and ISR at dose 1 injection in GLSI-100 treated subjects.

FIG. 4 shows injection site reactions (ISRs) by HER2 status in the subgroups of treated (GLSI-100) versus placebo (GM-CSF alone). The solid traces labeled “HER2 positive treated with GLSI-100” and “HER2 positive treated with GM-CSF alone” are the traces shown in solid lines of the FIG. 1 insert. FIG. 4 also includes the two additional traces for “HER2 low & intermediate treated with GLSI-100” and “HER2 low & intermediate treated with GM-CSF alone”. HER2+ and HER2 positive is equivalent to HER2 3+; HER2- and HER2 negative is equivalent to HER2 1+ and HER2 2+.

### DETAILED DESCRIPTION

The inventors have determined that GLSI-100 safely elicited a potent immune response, as evidenced by ISRs that correlate to and serve as a complement to immune response data such as DTH. The lower immune response of the generally trastuzumab treated HER2 positive control population, not evidenced in the low and intermediate HER2 expressors who did not receive trastuzumab, suggests a reduced immune state potentially related to prior trastuzumab exposure and/or HER2 positivity, which is reversed with the addition of GLSI-100. In HER2 positive (3+) patients, GP2 reverses a suppressed immune state, increases immune response, lowers cancer recurrence rates, and lengthens disease free survival.

The inventors have also determined that a peptide, such as a peptide of an oncogenic protein, reverses an immune state that is suppressed in a subject. The suppressed immune state can be due to, for instance, a prior treatment. A prior treatment can include, but is not limited to, surgery, chemotherapy, an antibody therapy that targets an oncogenic protein, or a combination thereof. The administration of the peptide, such as a peptide of an oncogenic protein, reverses immune suppression, causes a stronger immune response, lowers recurrence rates, and/or lengthens disease free survival. It has been demonstrated that if GM-CSF was administered to these patients without the peptide from the oncogenic protein, such as in the control group of a clinical study, the immune response to GM-CSF was suppressed when compared to patients who were not treated with an antibody to the oncogenic protein and who did not have high levels of expression of the oncogenic protein.

Examples of cancers that can be treated to reverse a suppressed immune state using the methods described herein include Her2/neu positive cancers such as breast cancer, ovarian cancer, gastric cancer, prostate cancer, lung cancer, colon and rectum cancer, urinary bladder and urinary corpus cancer, pancreas cancer, liver cancer, esophagus cancer, ovary cancer, and many other cancers, and may include many different types of proteins, such as oncoproteins, or combinations thereof. Other examples of cancers that can be analyzed using the methods described herein include, but are not limited to, the cancers listed in Table 1. The oncogenic proteins that correlate with different cancers are encoded by the oncogenes listed in Table 1. The peptide can be any set of consecutive amino acids from a protein, such as a protein that correlates with a cancer, e.g., an oncogenic protein. In one embodiment, the peptide is one that has therapeutic activity by activating the immune system, e.g., the peptide can be used to treat the subject to increase the immune response against a cancer and thus reduce an aspect of cancer and improve the health of the subject. For instance, a peptide can be one that reduces the recurrence of cancer in the subject by training the immune system to attack the cancer, including a recurrence caused by metastatic cancer. The therapeutic activity can result from the peptide or from the combination of the peptide with one or more other compounds. An example of a peptide with therapeutic activity is GP2, which is typically administered with GMCSF.

The methods can include determining a subject's immune response to a peptide, such as a protein correlated to a cancer, e.g., a peptide of an oncogenic protein. The methods include a subject that is a human, or an animal typically used as a model system for evaluating cancer-related treatments. In one embodiment, the subject is a human. In one embodiment, the subject can be a person that is at risk for a cancer, and the peptide used in the method is from an oncogenic protein that correlates with that cancer. A person that is at risk for a cancer includes, but is not limited to, a person who has been treated for a cancer and is in complete remission or in partial remission. A person that is at risk for a cancer also includes, but is not limited to, a person having one or more biomarkers, or a person with genetic risk for a cancer. Biomarkers are molecules, such as a gene or a protein, present in a subject and indicative of the progression of cancer. Examples of biomarkers include BRCA1, BRCA2, and prostate specific antigen (PSA). An example of a person with a genetic risk is one with a history of a cancer in relatives, such as first-degree relatives. In one embodiment, the subject can be one that has not previously been diagnosed as having a cancer. Such a person can be one that is at risk for a cancer, for instance, the subject can be one that has not been diagnosed as having a cancer but has one or more biomarkers and/or has a genetic risk of a cancer.

In another embodiment, the subject can be a person that was previously treated for a cancer, and the peptide used in the method is from a protein, such as a protein, for instance an oncogenic protein, that correlates with that cancer. In one embodiment, the subject is one that was previously treated for a cancer that was HER2/neu positive at a low, intermediate, or high level. HER2-positive cancers, such as breast cancers, are typically evaluated for HER2/neu expression and assigned an immunohistochemistry (IHC) score of 0 or 1+which can be referred to as HER2/neu low, HER2/neu 2+which can be referred to as HER2/neu intermediate, or HER2/neu 3+which can be referred to as HER2/neu high or 3+ or positive. “HER2 positive” or “HER2 3+” patients are defined by ASCO CAP guidelines (American Society of Clinical Oncology and College of American Pathologists), and also include fluorescence in situ hybridization (FISH) scores of about 2.0 or greater for HER2/neu gene expression. Methods for evaluating HER2/neu expression and assigning an IHC or FISH score are known in the art. In this embodiment, the cancer can be, but is not limited to, HER2/neu positive breast cancer, HER2/neu positive ovarian cancer, or HER2/neu positive gastric cancer, or any other HER2/neu positive cancer. In one embodiment, the cancer is HER2/neu positive breast cancer.

In one embodiment, the method can be used to treat a patient with a peptide, such as a peptide from an oncogenic protein. In one embodiment, the peptide is from a Her2/neu protein. The peptide can be from the cytoplasmic domain, the extracellular domain, or the transmembrane domain of a Her2/neu protein. In one embodiment, the peptide is from the transmembrane domain, and a non-limiting example of a peptide from the transmembrane domain is GP2 (Ile Ile Ser Ala Val Val Gly Ile Leu, SEQ ID NO:1). GP2, in combination with granulocyte-macrophage colony-stimulating factor (GM-CSF), is a therapy used to stimulate an immune response and to thus reduce cancer recurrence in breast cancer patients with tumors expressing any degree of HER2/neu (Patel et al., Five year median follow-up data from a prospective, randomized, placebo-controlled, single-blinded, multicenter, Phase IIb study evaluating the reduction of recurrences using HER2/neu peptide GP2-GM-CSF vs. GM-CSF alone after adjuvant trastuzumab in HER2 positive women with operable breast cancer. Presented at: 2020 San Antonio Breast Cancer Symposium; Dec. 8-11, 2020).

The immune response to a peptide, such as a peptide of a protein that correlates with a cancer, for instance an oncogenic protein, can be determined by any method. Immune response can be measured by, but is not limited to, delayed type hypersensitivity (DTH), injection site reaction (ISR), binding assays to T cells, functional cell-based assays for cytokine secretion such as Elispot, including an Elispot assay that quantifies T cell viability, measurement of antibody titers, and T cell assays including identification of specific T cells against any cancer target and including binding assays of T cells to HLA-peptide reagents that quantify peptide specific T cells. An immune response is considered a positive immune response if it is greater than the immune response of a suitable negative control. Thus, any increase in an immune response that is greater than a suitable negative control is a positive immune response. The skilled person can readily determine a negative control that can be used to determine if an immune response to a peptide, such as a peptide that correlates with a cancer, for instance an oncogenic protein. In one embodiment, the immune response is measured by testing a subject's DTH response to an intradermal injection of a peptide, such as a peptide that correlates with a cancer, for instance the protein at lower, similar, or higher quantities than a treatment dose. Methods for testing a DTH response are known in the art and routine. In one embodiment, induration (e.g., localized hardening) of the skin at the administration site is measured. When induration is determined the perpendicular diameter of the induration is typically measured.

Measurement can be by any suitable method, including calipers, ruler, or the ball point pen technique. The measurement can be expressed as the largest diameter of the skin reaction, or the orthogonal mean of the diameters is determined. An induration measurement of at least 3 mm, at least 5 mm, at least 7 mm or at least 10 mm or higher can be considered a positive DTH response. The DTH response of a subject can be measured from 24 to 72 hours, typically from 48 hours to 72 hours, after administration of the peptide at the chosen site

In those embodiments where the peptide is one that has therapeutic activity, the peptide can be used to determine an immune response before the peptide has been used for therapy. For instance, the peptide can be used to determine an immune response after the subject is diagnosed as having cancer, or after the subject has been treated, for instance by surgery or chemotherapy, for cancer. In one embodiment, GP2 is used as the peptide and it is administered for determining an immune response before the GP2 is used therapeutically. In another embodiment, GP2 is used as the peptide and it is administered for determining an immune response at approximately the same time the GP2 is administered therapeutically. Typically, the immune response that results from a test administered prior to the commencement of treatment or at approximately the same time as the commencement of treatment (e.g., before a subject can mount an immune response to an administered peptide) is referred to herein as a “baseline” response.

In those embodiments where the peptide is one that is not therapeutic, the peptide can be used to determine an immune response at any time, such as before the subject is diagnosed as having cancer, after the subject is diagnosed as having cancer, or after the subject has been treated, for instance by surgery or chemotherapy, for a cancer.

The amount of peptide administered to a subject to measure an immune response can vary and is not intended to be limiting. For instance, the range can be from 0.01 milligram/milliliter (mg/ml) to 1 mg/ml. When the peptide is a Her2/neu peptide, such as GP2, the amount can be 0.05 mg/ml to 0.4 mg/ml, and in one embodiment is 0.2 mg/ml. In one embodiment, the composition of peptide administered does not include an immunomodulatory agent such as GMCSF. The volume used can vary and is not intended to be limiting. For instance, the volume can be from 0.8 ml to 0.2 ml, and in one embodiment is 0.5 ml. The skilled person will recognize that a saline injection can be used as a negative control.

Following a positive immune response to a peptide, a subject can be treated with a therapy to reduce the cancer present, reduce the risk of recurrence of the cancer, or a combination thereof. The therapy can include, but is not limited to, antibody, antibody drug conjugate, checkpoint inhibitor, hormone therapy, tyrosine kinase inhibition, surgery, chemotherapy, or radiation therapy.

In some embodiments where the methods can be used to treat a subject, the method includes administering a peptide to the subject. The peptide can be at least at least 6 amino acids, at least 9 amino acids, or at least 12 amino acids in length. The peptide can be a peptide of an oncogenic protein, such as an oncogenic protein of Table 1. In one embodiment, the oncogenic protein is HER2/neu. In one embodiment, the peptide is GP2 or a derivative thereof. The amount of peptide administered to a subject for treatment can vary and is not intended to be limiting. For instance, the range can be from 0.01 milligram/milliliter (mg/ml) to 5 mg/ml. When the peptide is a Her2/neu peptide, such as GP2, the amount can be 0.2 mg/ml to 2 mg/ml, and in one embodiment is 1 mg/ml. The volume used can vary and is not intended to be limiting. For instance, the volume can be from 0.1 ml to 1 ml, and in one embodiment is 0.5 ml. The skilled person will recognize that a saline injection can be used as a negative control.

In one embodiment, treating the subject can include administration of an adjuvant. An example of an adjuvant is granulocyte macrophage colony-stimulating factor (GM-CSF). Examples of GM-CSF include but are not limited to a derivatized GM-CSF, an unglycosylated or partially unglycosylated GM-CSF, or a truncated GM-CSF. A GM-CSF can be made from any genetically modified cell line, e.g., recombinant, synthesized, or obtained from a natural source. In one embodiment, GM-CSF is sargramostim (Leukine®). The peptide and the adjuvant can be administered together in a composition, or administered independently and at the same time or different times. The administration can be intradermal, subcutaneous, intramuscular, or by inhalation.

The subject being treated can be one that has or had a cancer. The cancer can be a cancer listed in Table 1. In one embodiment, the cancer is a HER2/neu expressing cancer, such as breast cancer, ovarian cancer, gastric cancer, prostate cancer, lung cancer, colon and rectum cancer, urinary bladder and urinary corpus cancer, pancreas cancer, liver cancer, esophagus cancer, or ovary cancer. A HER2/neu expressing cancer is one that includes HER2/neu-expressing cells. The HER2/neu-expressing cells can have expression that is HER2/neu 3+ (also referred to as HER2/neu positive), HER2/neu intermediate, or HER2/neu low.

The subject can be one that has received prior treatment of the cancer. Examples of prior treatments include but are not limited to surgery, chemotherapy (e.g., a therapeutic agent or radiation), antibody therapy, targeted therapy, T cell therapy, RNA/DNA therapy, or a combination thereof. The prior treatment can be one that targets an oncogenic protein, such as an antibody therapy, targeted therapy, T cell therapy, or RNA/DNA therapy. In embodiments where the oncogenic protein is HER2/neu, an antibody can target any epitope on the HER2/neu protein. Examples of such antibodies includes but are not limited to trastuzumab (Herceptin®), ado-trastuzumab emtansine (Kadcyla™), fam-trastuzumab deruxtecan-nxki (Enhertu®), a trastuzumab biosimilar, pertuzumab (Perjeta®), or a derivative thereof. In some embodiments, an antibody or derivative thereof is an antibody-drug conjugate. Examples of antibody-drug conjugates include, but are not limited to, antibodies having one or more linked molecules, such as a chemotherapeutic agent. In an embodiment where the cancer is a cancer that includes HER2/neu expressing cells that are HER2/neu intermediate or HER2/neu low, the prior treatment includes an antibody therapy that targets HER2/neu. In an embodiment where the cancer is a cancer that includes HER2/neu expressing cells that are HER2/neu positive, the subject has not received prior treatment of the cancer that includes an antibody therapy that targets HER2/neu.

The methods to treat a subject can be used to reverse immune suppression in the subject, cause a stronger immune response in the subject, lower recurrence rate of the cancer in the subject, increase disease free survival in the subject, or a combination thereof. The methods can include determining a subject's immune state. A subject's immune state can be determined by any immune response assay to the peptide alone, the adjuvant alone, or the combination of both peptide and adjuvant. Examples of immune response assays include but are not limited to a delayed type hypersensitivity (DTH) immune response, an injection site reaction (ISR), an ELISPOT assay, an antibody titer assay, and a T cell assay. The subject's immune state can be determined before the treatment begins, at the time the treatment begins, after the treatment begins, or any combination thereof. In one embodiment, a subject's immune state is compared to the immune state of healthy or normal patients. A subject having a reduced immune response, e.g., a reduced ISR, compared to healthy or normal subjects is a suitable recipient of the treatment method. In one embodiment, a healthy or normal immune state has an ISR to a peptide, e.g., GP2, or an adjuvant, e.g., GM-CSF, of 50 millimeters (mm) or greater. In one embodiment, a low or suppressed immune state has an ISR to a peptide, e.g., GP2, or an adjuvant, e.g., GM-CSF, of less than 50 mm. In one embodiment, a low immune state has an ISR that is at least 40 mm less than the healthy or normal immune state ISR. A successful result of the treatment is restoration of the subject's immune system to a healthy or normal immune state. The treatment can also result in a stronger immune response in the subject, lowered recurrence rate of the cancer in the subject, increased disease free survival in the subject, or a combination thereof.

The present disclosure also provides the use of an adjuvant to determine a subject's immune state. The invention is defined in the claims. However, below there is provided a non-exhaustive listing of non-limiting exemplary aspects. Any one or more of the features of these aspects may be combined with any one or more features of another example, embodiment, or aspect described herein.

### Exemplary Aspects

Aspect 1. A method for treating a patient, comprising administering a peptide to a subject that has or had a cancer and has received prior treatment of the cancer, wherein the peptide reverses immune suppression, causes stronger immune responses, lowers cancer recurrence rates, and/or increases disease free survival.
Aspect 2. The method of Aspect 1, wherein the peptide is at least 6 amino acids.
Aspect 3. The method of Aspect 1 or 2, wherein the peptide is a peptide of an oncogenic protein.
Aspect 4. The method of any of Aspects 1 to 3, wherein the oncogenic protein is HER2/neu.
Aspect 5. The method of Aspect any of Aspects 1 to 4, wherein the prior treatment of the cancer comprises surgery, chemotherapy, antibody therapy, targeted therapy, T cell therapy, RNA/DNA therapy, or any other therapy that targets the oncogenic protein, or a combination thereof.
Aspect 6. The method of any of Aspects 1 to 5, wherein the antibody therapy, targeted therapy, RNA/DNA therapy, or T cell therapy targets an oncogenic protein.
Aspect 7. The method of any of Aspects 1 to 6, wherein the oncogenic protein is HER2/neu, and the antibody targets an epitope on the HER2/neu protein
Aspect 8. The method of any of Aspects 1 to 7, wherein the antibody is trastuzumab, a trastuzumab biosimilar, ado-trastuzumab emtansine, fam-trastuzumab deruxtecan-nxki, pertuzumab, or other antibodies that target an epitope on the HER2/neu protein, or a derivative thereof.
Aspect 9. The method of any of Aspects 1 to 8, wherein the antibody or derivative comprises a linked molecule or molecules, including potentially chemotherapeutic agents, such as an antibody-drug conjugate.
Aspect 10. The method of any of Aspects 1 to 9, wherein the peptide a HER2/neu derived peptide.
Aspect 11. The method of any of Aspects 1 to 10, wherein the peptide is GP2 or a derivative thereof.
Aspect 12. The method of any of Aspects 1 to 11, wherein the cancer comprises HER2/neu expressing cells.
Aspect 13. The method of any of Aspects 1 to 12, wherein the HER2/neu expressing cells are HER2/neu 3+, HER2/neu positive, HER2/neu intermediate, or HER2/neu low.
Aspect 14. The method of any of Aspects 1 to 13, wherein the cancer is breast cancer.
Aspect 15. The method of any of Aspects 1 to 14, wherein the subject had or has a HER2/neu positive cancer.
Aspect 16. The method of any of Aspects 1 to 15, wherein the cancer is a cancer comprising HER2/neu expressing cells that are HER2/neu intermediate or HER2/neu low, and wherein the prior treatment comprises an antibody therapy that targets HER2/neu.
Aspect 17. The method of any of Aspects 1 to 16, wherein the cancer is a cancer comprising HER2/neu expressing cells that are HER2/neu positive, and wherein the subject has not received prior treatment of the cancer comprising an antibody therapy that targets HER2/neu.
Aspect 18. The method of any of Aspects 1 to 17, wherein the treating further comprises administering an adjuvant.
Aspect 19. The method of any of Aspects 1 to 18, wherein the peptide and the adjuvant are administered together in a composition or administered independently and at the same or similar time.
Aspect 20. The method of any of Aspects 1 to 19, wherein determining the immune suppression comprises a delayed type hypersensitivity (DTH) immune response or injection site reaction (ISR) or any other immune response assay to the peptide or the adjuvant alone.
Aspect 21. The method of any of Aspects 1 to 20, wherein the peptide is a peptide of an oncogenic protein.
Aspect 22. The method of any of Aspects 1 to 21, wherein the oncogenic protein is HER2/neu.
Aspect 23. The method of any of Aspects 1 to 22, wherein the peptide of the oncogenic protein is GP2.
Aspect 24. The method of any of Aspects 1 to 23, wherein the subject had or has breast cancer, ovarian cancer, gastric cancer, cervical cancer, or any other HER2/neu expressing cancer.
Aspect 25. The method of any of Aspects 1 to 24, wherein determining the immune suppression comprises an immune response assay that can detect a subject's immune response to a peptide or the adjuvant alone.
Aspect 26. The method of any of Aspects 1 to 25, wherein the immune response assay is chosen from a DTH reaction, ISR, an ELISPOT assay, an antibody titer assay, and a T cell assay, or a combination thereof.
Aspect 27. The method of any of Aspects 1 to 26, wherein the testing compromises measurement of an injection site reaction to a therapeutic peptide against a cancer. Aspect 28. The method of any of Aspects 1 to 27, wherein the adjuvant alone is injected intradermally and the ISR is measured as a biomarker to determine the immune state of the patient.
Aspect 29. The method of any of Aspects 1 to 28, where the ISR measurements are compared to healthy or normal immune state patients, wherein a subject having a low immune state patient is treated to restore the immune system to a healthy or normal immune state.
Aspect 30. The method of any of Aspects 1 to 29, wherein the subject is treated with the peptide.
Aspect 31. The method of any of Aspects 1 to 30, where a healthy or normal immune state ISR is 50 mm or greater in diameter.
Aspect 32. The method of any of Aspects 1 to 31, where a low immune state ISR is less than 50 mm in diameter.
Aspect 33. The method of any of Aspects 1 to 32, where a low immune state ISR is 40 mm less in diameter than a healthy or normal immune state ISR.
Aspect 34. The method of any one of Aspects 1 to 34, wherein the adjuvant comprises granulocyte macrophage colony-stimulating factor (GM-CSF).
Aspect 35. The method of any of Aspects 1 to 34, wherein the GM-CSF is a derivatized GM-CSF, an unglycosylated or partially unglycosylated GM-CSF, or a truncated GM-CSF.
Aspect 36. The method of any of Aspects 1 to 35, wherein the GM-CSF is recombinant GM-CSF.
Aspect 37. The method of any of Aspects 1 to 36, wherein the GM-CSF is sargramostim.

### EXAMPLES

The present disclosure is illustrated by the following example. It is to be understood that the particular examples, materials, amounts, and procedures are to be interpreted broadly in accordance with the scope and spirit of the disclosure as set forth herein.

### Example 1

Background: Delayed type hypersensitivity (DTH) skin tests in the randomized, active-controlled, single-blinded, multicenter Phase IIb trial investigating GLSI-100 (GP2+GM-CSF) administered in the adjuvant setting to node-positive and high-risk node-negative breast cancer patients with tumors expressing any degree of HER2 (immuno-histochemistry [IHC] 1-3+) (NCT00524277) were analyzed. The trial enrolled HLA-A*02 patients randomized to receive GLSI-100 versus GM-CSF alone. The trial's primary objective was to determine if treatment with GLSI-100, a HER2-derived peptide, reduces recurrence rates.

Injection site reaction analysis in phase IIb trial of GP2

Methods: Patients were randomized and received GLSI-100 (500 micrograms (mcg) GP2 and 125 mcg GM-CSF) or control (GM-CSF) via 6 intradermal injections every 3-4 weeks for the first 6 months and 4 booster injections every 6 months. The magnitude of injection site reactions, which occurred in almost all patients, were assessed for the 10 doses administered by measuring the largest perpendicular diameters and the resulting orthogonal mean. DTH reactions were assessed in a similar manner at baseline and 6 months after treatment and have been shown to increase over time.

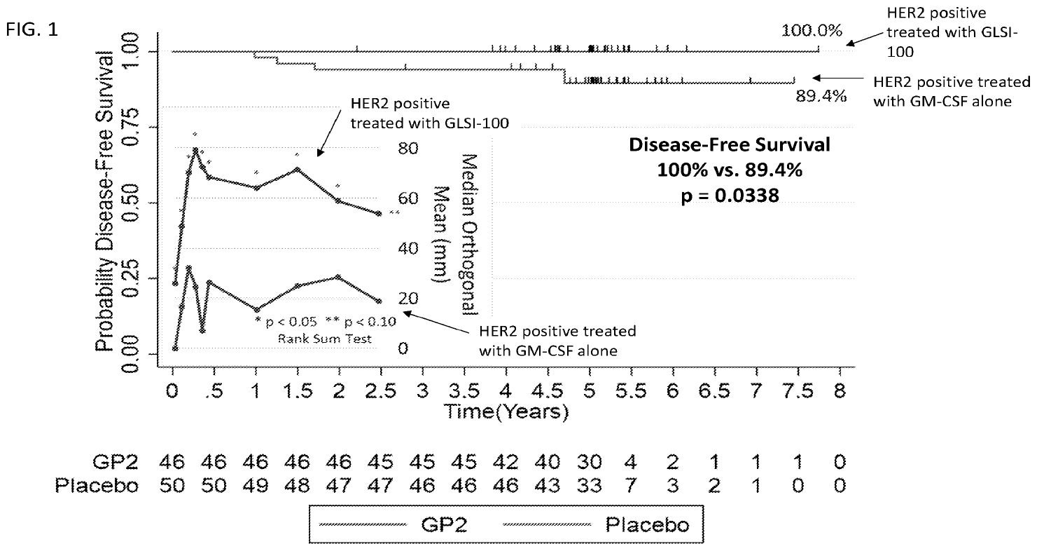

Results: The study enrolled 180 patients across 16 clinical sites with both HER2 3+positive and low and intermediate HER2 expressors (1-2+). After 5 years of follow-up, the Kaplan-Meier estimated 5-year disease free survival (DFS) rate in the 46 HER2 3+ patients treated with GLSI-100, if the patient completed the primary immunization series (PIS), was 100% versus 89.4% (95% CI: 76.2, 95.5%) in the 50 placebo patients treated with GM-CSF (p=0.0338) (FIG. 1). GLSI-100 was shown to be well tolerated with no serious adverse events (SAEs) deemed related to study medication and elicited a potent immune response measured by local skin tests and immunological assays. Injection site reactions were common, occurring in almost 100% of patients treated with either GLSI-100 or GM-CSF alone. Patients treated with GLSI-100 had statistically significantly larger injection site reactions compared to the size of reactions for patients treated with GM-CSF alone (p<0.05).

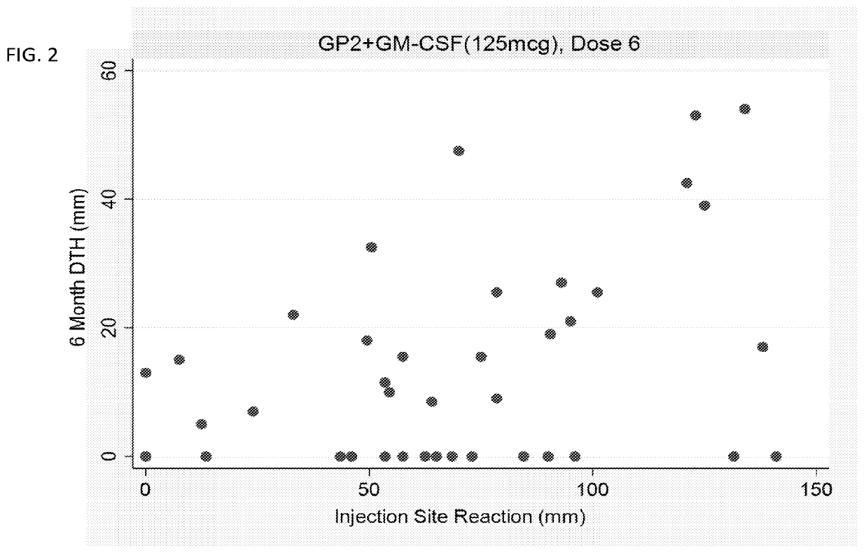

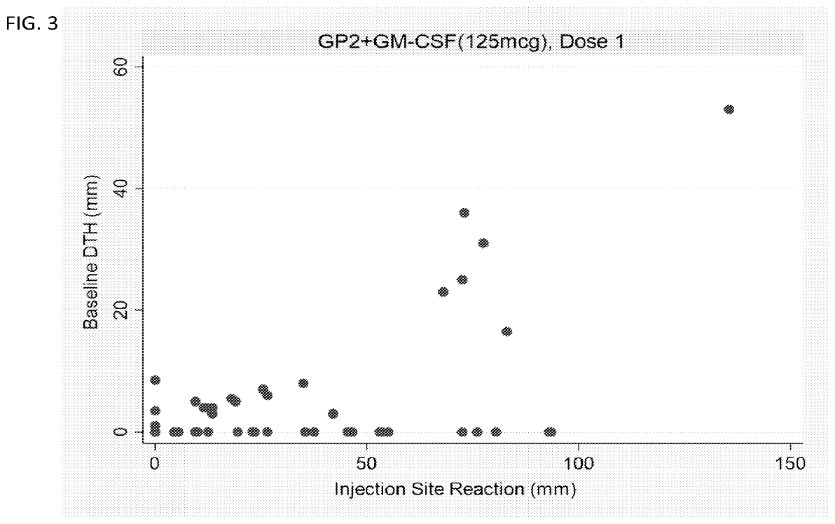

As shown in FIG. 2 and FIG. 3, DTH reactions at baseline and 6 months were consistently correlated with injection site reactions suggesting that injection site reactions may be interchangeable with immune response data (baseline ρ=0.6, p<0.001; 6mo ρ=0.4, ρ=0.009).

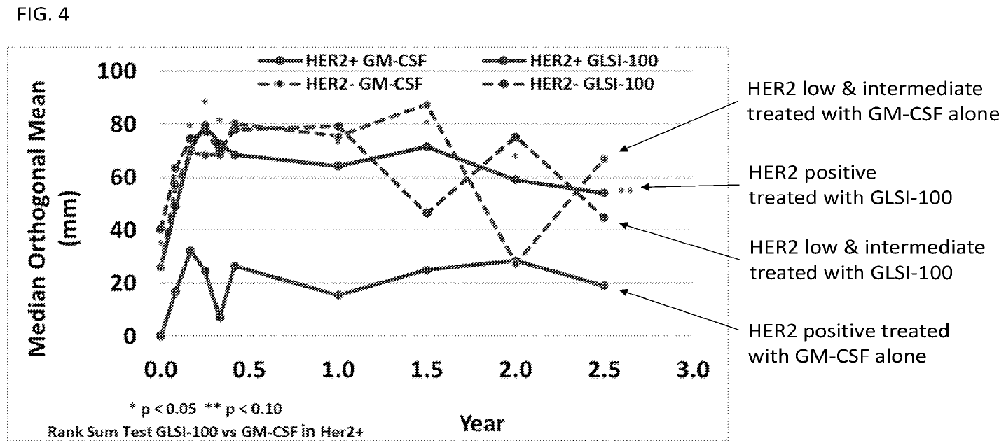

As shown in FIG. 4, after a single dose, the median orthogonal mean induration was 7.8 mm for GM-CSF patients and 34.0 mm for GLSI-100 patients (ρ=0.0036). Injection site reactions increased to 78 mm after the 4th injection but remained over 50 mm for the duration of the injection series for those treated with GLSI-100. The magnitude of injection site reactions for patients treated with GLSI-100 was similar across HER2 status. However, HER2 3+control patients (HER2+in FIG. 4), who were also treated with Herceptin®, had significantly smaller reactions to GM-CSF than HER2 1-2+control patients (HER2-in FIG. 4) who did not receive Herceptin®, with an average of 43.1 mm difference over all 10 doses. Trastuzumab treated HER2 3+control patients appeared to be in a suppressed immune state that was reversed by adding GP2 treatment, increasing injection site reactions by an average of 39.2 mm over all 10 doses. Trastuzumab treated HER2 3+ patients receiving GLSI-100 having the non-suppressed immune state (upper solid line of the FIG. 1 insert and upper solid line of FIG. 4) were the patients with 100% disease-free survival during the 5-year follow up (see FIG. 1). Conclusions: The treatment of HER2/neu positive patients, who received a standard course of trastuzumab after surgery, with GLSI-100 (GP2+GM-CSF), safely elicited a potent immune response, as evidenced by larger injection site reactions, reduced recurrence rates to 0%, and increased diseasefree survival to 100% over 5 years of follow-up. The injection site reactions of HER2 positive patients treated with GLSI-100 were significantly larger than those of the GM-CSF alone control group, providing further evidence of specific GP2 immune response produced by treatment with GLSI-100. It has been demonstrated that if GM-CSF is administered to control patients without the peptide from the oncogenic protein, the immune response to GM-CSF is lower when compared to patients who are not treated with an antibody to the oncogenic protein and who do not have high levels of expression of the oncogenic protein, immune state is suppressed, recurrence rate is higher, and disease free survival is lower, which are all reversed by the addition of the GP2.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The disclosure is not limited to the exact details shown and described, for variations obvious to one skilled in the art will be included within the disclosure defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A method for treating a patient, comprising administering a peptide to a subject that has or had a cancer and has received prior treatment of the cancer, wherein the peptide reverses immune suppression, causes stronger immune responses, lowers cancer recurrence rates, and/or increases disease free survival.

2. The method of claim 1, wherein the peptide is at least 6 amino acids.

3. The method of claim 1, wherein the peptide is a peptide of an oncogenic protein.

4. The method of claim 3, wherein the oncogenic protein is HER2/neu.

5. The method of claim 1, wherein the prior treatment of the cancer comprises surgery, chemotherapy, antibody therapy, targeted therapy, T cell therapy, RNA/DNA therapy, or any other therapy that targets the oncogenic protein, or a combination thereof.

6. The method of claim 5, wherein the antibody therapy, targeted therapy, RNA/DNA therapy, or T cell therapy targets an oncogenic protein.

7. The method of claim 6, wherein the oncogenic protein is HER2/neu, and the antibody targets an epitope on the HER2/neu protein

8. The method of claim 7, wherein the antibody is trastuzumab, a trastuzumab biosimilar, ado-trastuzumab emtansine, fam-trastuzumab deruxtecan-nxki, pertuzumab, or other antibodies that target an epitope on the HER2/neu protein, or a derivative thereof.

9. The method of claim 7, wherein the antibody or derivative comprises a linked molecule or molecules, including potentially chemotherapeutic agents, such as an antibody-drug conjugate.

10. The method of claim 4, wherein the peptide a HER2/neu derived peptide.

11. The method of claim 10, wherein the peptide is GP2 or a derivative thereof.

12. The method of claim 4, wherein the cancer comprises HER2/neu expressing cells.

13. The method of claim 12, wherein the HER2/neu expressing cells are HER2/neu 3+, HER2/neu positive, HER2/neu intermediate, or HER2/neu low.

14. The method of claim 1, wherein the cancer is breast cancer.

15. The method of claim 1, wherein the subject had or has a HER2/neu positive cancer.

16. The method of claim 1, wherein the cancer is a cancer comprising HER2/neu expressing cells that are HER2/neu intermediate or HER2/neu low, and wherein the prior treatment comprises an antibody therapy that targets HER2/neu.

17. The method of claim 1, wherein the cancer is a cancer comprising HER2/neu expressing cells that are HER2/neu positive, and wherein the subject has not received prior treatment of the cancer comprising an antibody therapy that targets HER2/neu.

18. The method of claim 1, wherein the treating further comprises administering an adjuvant, wherein the adjuvant comprises granulocyte macrophage colony-stimulating factor (GM-CSF).

19. The method of claim 18, wherein the peptide and the adjuvant are administered together in a composition or administered independently and at the same or similar time.

20. The method of claim 1, wherein determining the immune suppression comprises a delayed type hypersensitivity (DTH) immune response or injection site reaction (ISR) or any other immune response assay to the peptide or the adjuvant alone.

21-37. (canceled)


SEQUENCE LISTING (1 sequence)

SEQ ID NO: 1 - protein, 9 aa, synthetic construct
IISAVVGIL